(19) United States
(12) Patent Application Publication
(10) Pub. No.: US 2006/0057718 A1
(43) Pub. Date: Mar. 16, 2006

(54) Stem cell and progenitor cell expansion

(75) Inventors: Tannishtha Reya, Chapel Hill, NC (US); Mweia Uqoezwa, Raleigh, NC (US); Qiju Wu, Durham, NC (US); Leah DiMascio, Chapel Hill, NC (US)

Correspondence Address: Medlen & Carroll, LLP;Suite 350, 101 Howard Street, San Francisco, CA, 94105, US

(73) Assignee: Duke University, Durham, NC (US)

(21) Appl. No.: 11/115,679

(22) Filed: Apr. 27, 2005

Related U.S. Application Data
(60) Provisional application No. 60/565,806, filed on Apr. 27, 2004.

(51) Int. Cl. C12N 5/08

(52) U.S. Cl. 435/366; 435/370; 435/372; 435/368

(57) ABSTRACT

The present invention provides compositions and methods for the expansion of stem cells and progenitor cells with adiponectin, adiponectin variants, or other molecules that activate adiponectin receptors and signaling, whereby the stem or progenitor cells retain their pluripotential phenotype after expansion. In certain embodiments, the present invention provides adiponectin-expanded stem cells used to treat disease (e.g. used in bone marrow transplants) or allows direct delivery in vivo to enhance hematopoietic recovery.

The present Application claims priority to U.S. Provisional Application Ser. No. 60/565,806, filed Apr. 27, 2004, herein incorporated by reference.

The present application was funded in part with government support under grant number R01DK 063031-01 from the National Institutes of Health. The government has certain rights in this invention.

### FIELD OF THE INVENTION

The present invention relates to compositions and methods for inducing the proliferation and expansion of stem cells and progenitor cells with adiponectin, adiponectin variants, or other molecules that activate adiponectin receptors, whereby the stem or progenitor cells retain their pluripotential phenotype after expansion. In certain embodiments, the present invention provides adiponectin-expanded stem cells used to treat disease (e.g. used in bone marrow transplants).

### BACKGROUND OF THE INVENTION

A major goal in stem cell transplantation is the selection and ex vivo expression of hematopoietic stem cells (HSC). Hematopoietic stem cells are rare primitive blood cell progenitors that have the capacity to differentiate, so as to give rise to various morphologically recognizable precursors of blood cell lineages. These precursors are immature blood cells that cannot self-replicate and must differentiate into mature blood cells including the erythroid, lymphoid, and myeloid cells. Within the bone marrow microenvironment, the stem cells self-proliferate and actively maintain continuous production of all mature blood cell lineages throughout life.

Bone marrow (BM) transplantation is being increasingly used in humans as an effective therapy for an increasing number of diseases, including malignancies such as leukemias, lymphoma, myeloma and selected solid tumors as well as nonmalignant conditions such as severe aplastic anemias, immunologic deficiencies, and inborn errors of metabolism. The availability of stem cells would be extremely useful in bone marrow transplantation including the augmentation of bone marrow transplantation (BMT) and the replacement of BMT, as well as transplantation of other organs in association with the transplantation of bone marrow. Stem cells are also important targets for gene therapy, where the inserted genes promote the health of the individual into whom the stem cells are transplanted. However, expansion of bona fide, long-term reconstitutive HSC (LT-HSC), capable of differentiation into the lymphoid and myeloerythroid series, and capable of long term engraftment, have proven quite challenging. Current methods of HSC expression have not proven reliable. Thus, the art is in need of methods and compositions for efficient expansion of HSCs.

### SUMMARY OF THE INVENTION

The present invention provides compositions and methods for the expansion of stem cells and progenitor cells with adiponectin, adiponectin variants, or other molecules that activate adiponectin receptors, whereby the stem or progenitor cells retain their pluripotential phenotype after expansion. In certain embodiments, the present invention provides adiponectin-expanded stem cells used to treat disease (e.g. used in bone marrow transplants).

In some embodiments, the present invention provides methods for in vitro expansion of stem or progenitor cells comprising: a) providing: i) stem or progenitor cells; and ii) a composition comprising adiponectin or an adiponectin variant (or other molecule that activates an adiponectin receptor); and b) culturing the cells with the composition under conditions that promote the expansion of the cells. In certain embodiments, the culturing generates a population of expanded stem cells. In some embodiments, the culturing is under conditions whereby the cells retain their pluripotential phenotype after expansion. In certain embodiments, the method further comprises providing a host and transplanting expanded cells produced according to step b) into the host. In other embodiments, the cells are provided from the host.

In particular embodiments, the present invention provides kits comprising; a) a composition comprising adiponectin or an adiponectin variant (or other molecule that activates an adiponectin receptor); and b) written instructions for using the composition to expand stem cells or progenitor cells.

In some embodiments, the present invention provides compositions comprising; a) ex vivo sorted stem or progenitor cells; and b) adiponectin or an adiponectin variant (or other molecule that activates an adiponectin receptor) present in a concentration between 50 and 150 ng/ml. In further embodiments, the compositions further comprise SLF, Wnt3A, FLT, TPO, a carrier, or any combination thereof. In particular embodiments, the compositions further comprise lipids. In some embodiments, the compositions further comprise fatty acids. In other embodiments, the composition comprises at least one additional HSC growth factor. Examples of suitable, additional growth factors include, but are not limited to:beta-catenin, c-kit ligand, LIF, IL-11, as well as additional growth factors found in the following publications: U.S. Pat. Nos. 5,861,315; 5,668,104; 5,556,954; 5,270,458; 6,465,249; and PCT publications WO 99/16864, WO 99/61589, WO 99/40180, WO 99/65299, and WO 00/06704, all of which are herein incorporated by reference in their entireties. In preferred embodiments, the at least one additional growth factor is present at a concentration between 5 ng/ml and 50 ng/ml. In certain embodiments, the stem or progenitor cells are expanded with adiponectin (or variant thereof) on a feeder layer.

In certain embodiments, the stem or progenitor cells are expanded with a molecule that activates an adiponectin receptor (e.g. AdipoR1 or AdipoR2, see Yamauchi et al., Nature, 2003, Jun. 12; 423(6941):762-9, herein incorporated by reference). Molecules able to activate an adiponectin receptor such as AdipoR1 or R2 may be found, for example, by screening a library of molecules against these receptors to find those that bind, and then conducting a cell based assay using the molecules that bind to find those that activate the receptor.

In some embodiments, the stem or progenitor cells are expanded with a molecule that activates a downstream adiponectin pathway molecule, such as a molecule that promotes phosphorylation of AMP activated protein kinase (e.g. AMPK activator 5-aminoimidazole-4-carboxamide ribonucleoside (AICAR); or METFORMIN, or AVANDIA (rosiglitazone maleate), or derivatives of any of these compounds or similar compounds). In other embodiments, the stem or progenitor cells function is modulated with a molecule that inhibits a downstream adiponectin pathway molecule, such as a molecule that inhibits acetyl CoA carboxylase. In other embodiments, the stem or progenitor cells are expanded with a molecule that increases fatty acid beta-oxidation in the stem or progenitor cells or by direct addition of lipids in the presence or absence of adiponectin.

In particular embodiments, the stem cells are adult stem cells. In other embodiments, stem cells are embroyonic stem cells. In some embodiments, the stem cells are selected from the group consisting of: HSCs, stromal stem cells, neuronal crest stem cells, mesenchymal stem cells, pulmonary stem cells and hepatic stem cells. In some embodiments, the stem cells are non-human. In preferred embodiments, the stem cells are human stem cells.

In other embodiments, the adiponectin or adiponectin variant is recombinant. In some embodiments, the stem cells are ex vivo sorted stem cells. In other embodiments, the adiponectin or adiponectin variant is present at a concentration of between 80 and 120 ng/ml. In particular embodiments, the adiponectin or adiponectin variant is present at a concentration of between 90 and 110 ng/ml.

In other embodiments, the stem or progenitor cells comprise a heterologous gene encoding a marker. In certain embodiments, the stem cells or progenitor cells comprise a heterologous gene of interest (e.g. a gene that encodes a protein of interest when the cells are transplanted in a host).

### DESCRIPTION OF THE FIGURES

FIG. 1 shows that adiponectin (a) induces enhanced HSC survival and proliferation in vitro compared to control conditions (b).

FIG. 2 shows adiponectin synergizes with other stem cell growth factors to induce HSC growth in vitro.

FIG. 3 shows the addition of adiponectin increases the growth of HSCs. Two days after plating, the well with adiponectin (a) is growing better than the well without adiponectin (b). The HSCs are proliferating much more with the addition of Adiponectin (c) than the ones without adiponectin (d) after six days culture in vitro.

FIG. 4 shows HSCs cultured with adiponectin retain characteristics of HSCs and the majority are Lin negative, Thy-1.1 positive (a), Sca-1 positive, and C-Kit positive (b).

FIG. 5 shows the results from Example 2, which describes assays used to determine the levels of Adiponectin Receptor expression levels in proliferating HSCs compared to quiescent cells. In particular, FIG. 5a shows that AdipoR1 is upregulated approximately 3-fold in proliferating KTLS cells, and FIG. 5b shows a similar pattern with AdipoR2.

FIG. 6 shows that culture with adiponectin selectively enhances the proliferation of long term HSCs in vitro. Wild type Purified KTSLin⁻ (A), KTSLin^{-/lo} (B) and were cultured with Tpo and SCF in the presence (solid line) or absence (dashed line) of adiponectin for an 8 day period during which total cell number was analyzed. (C) KTSLin⁻ cells from Bcl2 transgenic mice were cultured with SCF alone in the presence (solid line) or absence (dashed line) of adiponectin for 8 days during which total cell number was analyzed.

FIG. 7 shows cells cultured in the presence of adiponectin retain immature cell characteristics. A) Mature lineage marker expression of wild type purified KTSLin⁻ cells cultured in the presence or absence of Adiponectin. B) Colony forming ability of KTSLin⁻ cells cultured for 3 days in the presence or absence of Adiponectin and subsequently cultured in methylcellulose media.

FIG. 8 shows that inhibition of adiponectin signaling leads to decreased cell counts in vitro due to decreased proliferation. siRNA was designed against AdipoR1 and an unrelated control in a lentiviral vector expressing GFP. A) 7F2 osteoblast precursor cells were infected with lentiviral R1 siRNA or an unrelated control and expression of R1 was examined in GFP+ cells by qPCR. B) KTLS cells were infected with siRNA against AdipoR1, or an unrelated control. R1 expression was examined in GFP+ cells by qPCR. C) KTLS cells infected with R1 or control siRNA were cultured in a rich growth media (5% FBS, 30 ng/ml SCF and Flt3 ligand) for 4 days at which time total cell count was determined.

### GENERAL DESCRIPTION OF THE INVENTION

The present invention provides compositions and methods for inducing the proliferation and/or expansion of stem cells and progenitor cells with adiponectin, adiponectin variants, or other molecules that activate adiponectin receptors, whereby the stem or progenitor cells retain their pluripotential phenotype after expansion. In certain embodiments, the present invention provides expanded stem cells used to treat disease (e.g. used in bone marrow transplants), or enhances hematopoeitic recovery by direct delivery in vivo.

Primitive stem cells (HSC) continuously expand in vivo at a slow rate. However, ex vivo expansion of stem cells with the preservation of stem cell quality has not been adequately achieved. The roles of growth factors, cytokines, transmembrane signaling and microenvironment have been widely studied in an attempt to impel HSCs to expand in vitro without differentiating, while improving hematopoietic recovery in vivo using in vitro-propagated grafts.

In vitro expansion/self renewal of high-quality stem cells (e.g. HSCs) would be highly valuable for use in bone marrow transplantation and would solve many of the problems associated with hematologic cancer and other solid cancers. Much work has been invested in research designed to culture HSC but adequate HSC expansion agents have not been identified prior to the present invention.

In some embodiments, the present invention provides enhanced methods for ex vivo expansion of stem cells (e.g. hematopoietic stem cells). For example, in some embodiments, cells are cultured in the presence of adiponectin and adiponectin variants.

The present invention also provides compositions and methods for cell transplantation. Adiponectin treated—expanded stem cells (or progenitor cells) are introduced into a host. Transplanted cells find may uses. For example, the adiponectin treated-cells may be provided for therapeutic uses. The cells may also be provided with a gene of interest, which expresses a desired factor in the host (e.g., therapeutic protein, antisense construct, reporter, etc.). The cells may also be transplanted to analyze cell maintenance and expansion in vivo. Such embodiments may be used to identify, characterize, and optimize factors (e.g., drugs) that regulate cell expansion or maintenance or other cellular processes.

### DEFINITIONS

To facilitate an understanding of the present invention, a number of terms and phrases are defined below:

As used herein, the “expansion” of a stem cell indicates that there is an increase in the absolute number of stem cells (e.g., during the culturing of the cells). Analogously, a stem cell that has undergone such expansion has been “expanded.”

As used herein, the term “transplant” refers to tissue used in grafting, implanting, or transplanting, as well as the transfer of tissues from one part of the body to another, the return of cells to the original donor (autologous transplants) or the transfer of tissues from one individual to another, or the introduction of biocompatible materials into or onto the body. The term “transplantation” refers to the grafting of tissues from one part of the body to another part, or to another individual.

As used herein, the term “engrafting” a stem cell (e.g., an adiponectin expanded stem cell) refers to placing the stem cell (e.g. HSC) into an animal (e.g., by injection), wherein the stem cell persists in vivo. This can be readily measured, for HSCs, by the ability of the HSC to contribute to ongoing blood formation.

As used herein, the term “stem cell” or “undifferentiated cell” refers to self-renewing cells that are capable of giving rise to phenotypically and genotypically identical daughters as well as at least one other final cell type (e.g., terminally differentiated cells). Stem cells include, but are not limited to, hematopoietic stem cells and progenitor cells derived therefrom (see U.S. Pat. No. 5,061,620, herein incorporated by reference); neural crest stem cells; embryonic stem cells; mesenchymal stem cells; mesodermal stem cells; stromal stem cells, pulmonary epithelial stem cells, hepatic stem cells, and other stem cells.

As used herein, the term “host” refers to any warm blooded mammal, including, but not limited to, humans, non-human primates, rodents, and the like, which is to be the recipient of a particular treatment. Typically, the terms “host” and “patient” are used interchangeably herein in reference to a human subject.

As used herein, the term “carrier” refers to a diluent, adjuvant, excipient, or vehicle with which a compound is administered. Such pharmaceutical carriers can be sterile liquids, such as water and oils, including those of petroleum, animal, vegetable, or synthetic origin, such as peanut oil, soybean oil, mineral oil, sesame oil and the like. Water or aqueous solution saline solutions and aqueous dextrose and glycerol solutions are preferably employed as carriers, particularly for injectable solutions.

As used herein, the term “therapeutically effective amount” refers to an amount sufficient to reduce by a least about 15 percent, preferably by at least 50 percent, more preferably by at least 90 percent, and most preferably prevent, a clinically significant deficit in the activity, function, and/or response of a host. Alternatively, a therapeutically effective amount is sufficient to cause an improvement in a clinically significant condition in a host.

As used herein, the term “heterologous gene” refers to a gene encoding a factors that is not in its natural environment. For example, a heterologous gene includes a gene from one species introduced into another species. A heterologous gene also includes a gene native to an organism that has been altered in some way (e.g., mutated, added in multiple copies, linked to non-native regulatory sequences, etc). Heterologous genes are distinguished from endogenous genes in that the heterologous gene sequences are typically joined to DNA sequences that are not found naturally associated with the gene sequences in the chromosome or are associated with portions of the chromosome not found in nature (e.g., genes expressed in loci where the gene is not normally expressed). Heterologous genes may be introduced into stem or progenitor cells through molecular biology manipulation. The coding sequence of the heterologous gene is operatively linked to an expression control sequence. Generally a heterologous gene is first placed into a vector.

As used herein, the term “gene-modified stem cell” refers to a stem cell that has been transduced by a heterologous gene.

The term “recombinant DNA molecule” as used herein refers to a DNA molecule that is comprised of segments of DNA joined together by means of molecular biological techniques (e.g. a human adiponectin gene present on a plasmid).

The term “recombinant protein” or “recombinant polypeptide” as used herein refers to a protein molecule that is expressed from a recombinant DNA molecule (e.g. human adiponectin expressed by E. coli cells containing a plasmid with the human adiponectin gene).

As used herein, the term “in vitro” refers to an artificial environment and to processes or reactions that occur within an artificial environment. In vitro environments can consist of, but are not limited to, test tubes and cell culture. The term “in vivo” refers to the natural environment (e.g., an animal or a cell) and to processes or reaction that occur within a natural environment.

### DETAILED DESCRIPTION OF THE INVENTION

The present invention provides enhanced methods for the expansion of stem and progenitor cells using adiponectin and adiponectin variants (as well as other molecules that activate an adiponectin receptor). In some embodiments of the present invention, stem cells (e.g. HSCs) are cultured in the presence of adiponectin or adiponectin variants. In preferred embodiments, the cells are also cultured in an additional growth factor (e.g. SCF, Tpo, or FLT-3). The stem cells may further comprises one or more other heterologous genes of interest (e.g., a therapeutic gene or a reporter gene). In certain preferred embodiments, the stem cells further comprise a selectable marker or a detectable marker (e.g., to allow detection or isolation of transfected cells and/or to allow monitoring of cells in vivo).

The present invention further provides methods for the transplantation of expanded adiponectin-treated stem cells (or progenitor cells) into host organisms. Cells may be transplanted for therapeutic applications and/or for expression of a transgene, or may be transplanted to monitor cell localization and maintenance in a host (e.g., over time or in response to further treatment with drugs).

Certain preferred embodiments of the present invention are described in detail below. The present invention is not limited to these particular described embodiments. The description is provided in the following section: I) Identification and in vitro isolation of stem cells; II) Adiponectin and adiponectin variant treatment of stem cells; III) Heterologous gene expression; and IV) Transplantation.

I. Identification and In Vitro Isolation of Stem Cells

Stem cells, such as HSCs, have been isolated and enriched from suitable sources using transmembrane glycoproteins (e.g., the CD34 molecule that is expressed on human hematopoietic stem cells and on committed progenitor cells [HPC]). Dye exclusion, (e.g., Rh-123) has also been used to isolate stem cells (Goodell et al., Nature Med., 3:1337 [1997]). Stem cells for use in the present invention are not limited by the method of isolation.

Bone marrow cells can be obtained from any number of sources from an animal, including a human subject. For example, the cells can be harvested from iliac bone marrow. Alternatively, stem cells can be obtained from umbilical chord cells. Another source for stem cells (e.g. HSCs) is from circulating fetal blood cells. In addition, a human subject, for example, can be treated with a cytotoxic drug and/or a stem cell stimulating cytokine (e.g., G-CSF). Mononuclear cells can then be collected by leukophoresis and the hematopoictic stem cells can be isolated from the peripheral blood cells by their selective binding to an antibody raised against CD34.

II. Adiponectin and Adiponectin Variants for Stem Cell Expansion

The present invention provides for the expansion of stem cells and progenitor cells through the use of adiponectin and adiponectin variants. For example, stem cells may be cultured in vivo with different concentrations of adiponectin in order to cause the stem cells to proliferate and/or increase the survival rate of these cells. Examples of suitable concentrations include, but are not limited to, between 1 and 500 ng/ml of adiponection or adiponectin variant. These cells may also be cultured with SCF, FLT-3 or Tpo.

Human adiponectin is a peptide that is 244 amino acids in length (see Maeda et al., Biochem, Biophys. Res. Comm., 221:286-289; 1996; and Saito et al., Gene, 229:67-73, 1999, both of which are herein incorporated by reference). The present invention may also be practiced with adiponectin variants, such deletion mutants, sequence change variants, truncated versions of adiponectin, etc. Example of adiponectin variants (and adiponectin variants) are provided in Pat. Pub. US20040023854A1 to Cooper et al., and WO03055916 to Rasmussen et al., both of which are herein incorporated by reference in their entirities. Preferred variants may be identified by screening them against stem cells (e.g. as shown in Example 1) to identify the variants that are able expand stem cells the best.

III. Modulating Downstream Adiponectin Pathway Molecules

In some embodiments, the stem or progenitor cells are expanded with a molecule that activates a downstream adiponectin pathway molecule, such as a molecule that promotes phosphorylation of AMP activated protein kinase (e.g. AMPK activator 5-aminoimidazole-4-carboxamide ribonucleoside (AICAR); or METFORMIN, or AVANDIA (rosiglitazone maleate), or derivatives of any of these compounds or similar compounds). In other embodiments, the function of stem or progenitor cells are modulated with a molecule that inhibits a downstream adiponectin pathway molecule, such as a molecule that inhibits acetyl CoA carboxylase. In other embodiments, the stem or progenitor cells are expanded with a molecule that increases fatty acid beta-oxidation in the stem or progenitor cells, or by addition of lipids in the extracellular environment in the presence or absence of adiponectin. Compounds that modulate downstream adiponectin pathway molecules, such as compounds that promote phosphorylation of AMP activated protein kinase (see Winder and Hardie, Am J Physiol. 1999 July;277(1 Pt 1):E1-10, herein incorporated by reference, for a review of AMPK activity), can be found by testing such candidate compounds in assays similar to those described in the Examples below (e.g. substitute the candidate compound for adiponectin in the Examples below). In this regard, candidate compounds (as well as adiponectin variants) can be identified by screening individual compounds or libraries of compounds.

IV. Heterologous Gene Expression

Certain embodiments of the present invention employ heterologous genes in the stem cells. In some embodiments, the heterologous gene is a gene of interest such as a therapeutic gene or a reporter gene.

Vectors for ex vivo administration of a gene encoding a heterologous gene may be introduced via any strategy. Vectors can be introduced to transduce the desired host cells ex vivo by methods including, but not limited to, beta-catenin (see U.S. Pat. No. 6,465,249, herein incorporated by reference), transfection, electroporation, microinjection, cell fusion, DEAE dextran, calcium phosphate precipitation, lipofection, use of a gene gun, viral vector, DNA vector transporter, and the like. Any gene of interest may be expressed in the stem cells. Examples of genes that find use with the present invention include, but are not limited to, therapeutics genes, reporter genes, and selectable markers. Examples of therapeutic genes include, but are not limited to, hematopoietic stem cells encoding human β-globin to treat thalassemia and cells encoding glucocerebrosidase to treat Gaucher's disease. Reporter genes may be expressed, for example, to monitor the expansion, differentiations, and maintenance of cells in vivo. Selectable markers may be used, for example, to select cells that have undergone a successful transfection event.

V. Transplantation

The present invention provides cells and methods for transplantation into host organisms. Transplantation of adiponectin-treated and expanded stem cells into a host may be used, for example, to provide a source of stem cells for generating and supplying differentiated products, to express a gene of interest, and to detect and characterize cell expansion and differentiation in vivo (e.g., to provide detectable cells for testing drugs that influence cell expansion, differentiation, and cell fate in vivo). As such, both human and non-human animal hosts find use in the present invention.

In preferred embodiments, where cells are to used for therapeutic purposes, the stem cell (or progenitor cells) is preferably obtained from the subject in need of treatment, and then after expansion, the resulting adiponectin-treated stem cell is placed back into the host (See, WO 99/61589 for methods of reintroduction into hosts, herein incorporated by reference in its entirety).

Experimental

The following example serves to illustrate certain preferred embodiments and aspects of the present invention and is not to be construed as limiting the scope thereof.

### EXAMPLE 1

### Adiponectin Enhances Survival and Proliferation of HSCs

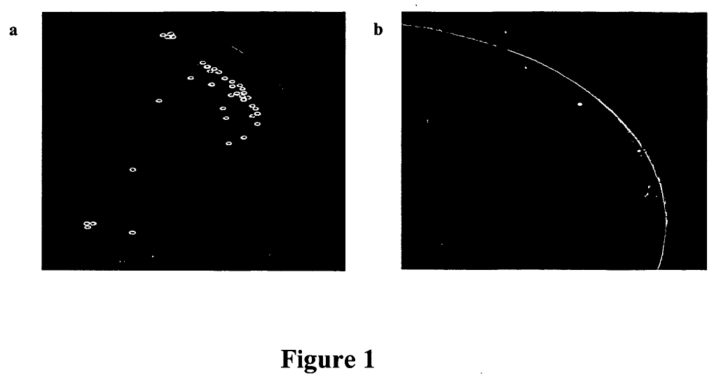

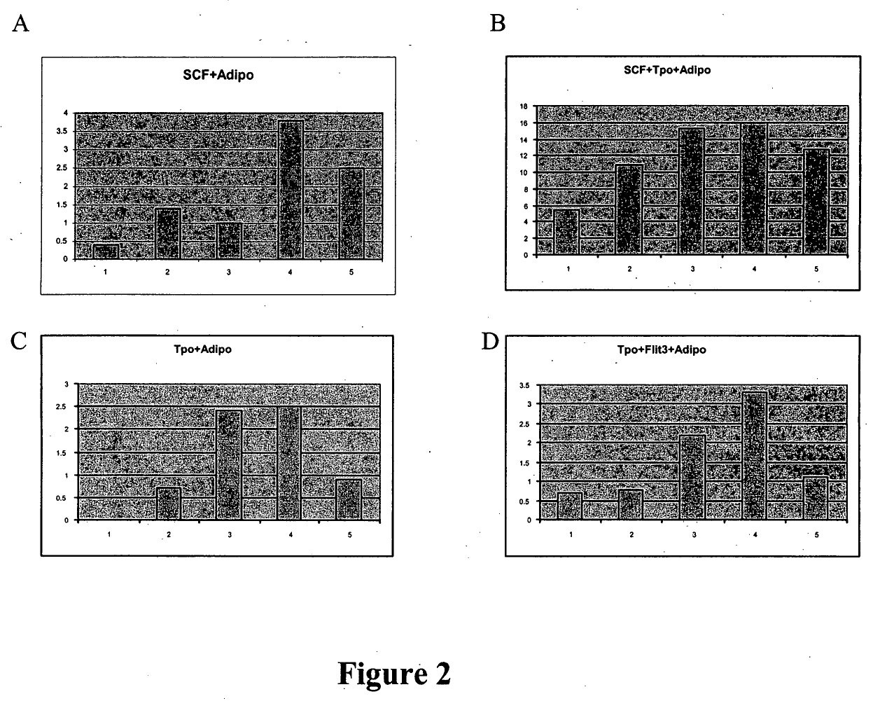

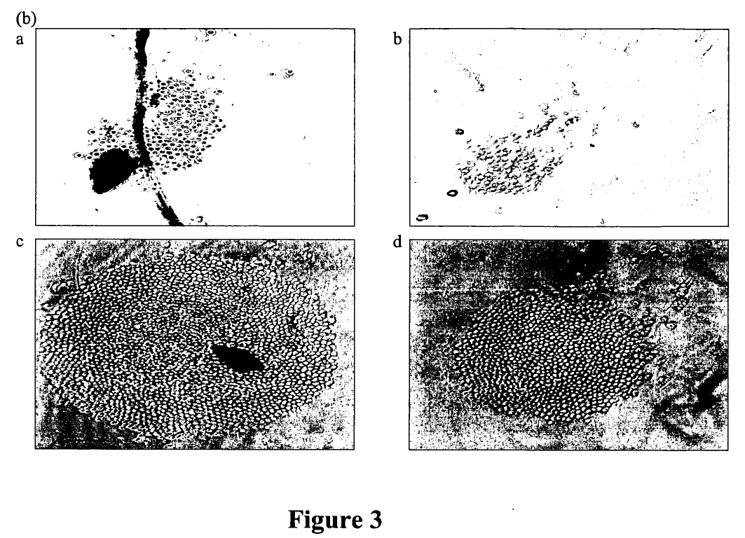

This example demonstrates that adiponectin enhances survival and proliferation of HSCs. HSCs sorted via fluorescence-activated sorting (FACS) using cell-surface markers (c-Kit+, Thy-1.1^{lo}, Lin^{-/lo}, and Sea-1+ (KTLS) cells) were plated at a density of 20 cells per well. A range of adiponectin concentrations from 0 to 500 ng/ml in the presence or absence of 15 ng/ml SCF, 30 ng/ml FLT-3, 30 ng/ml Tpo were tested to determine if adiponectin could enhance the survival and the proliferation of HSCs. As shown in FIGS. 1 and 2, adiponectin can drive the proliferation of HSCs in synergy with SLF as well as other growth factors. Its activity is concentration dependent with a peak at 100 ng/ml (FIG. 2). FIG. 3 shows the addition of adiponectin increases the growth of HSCs.

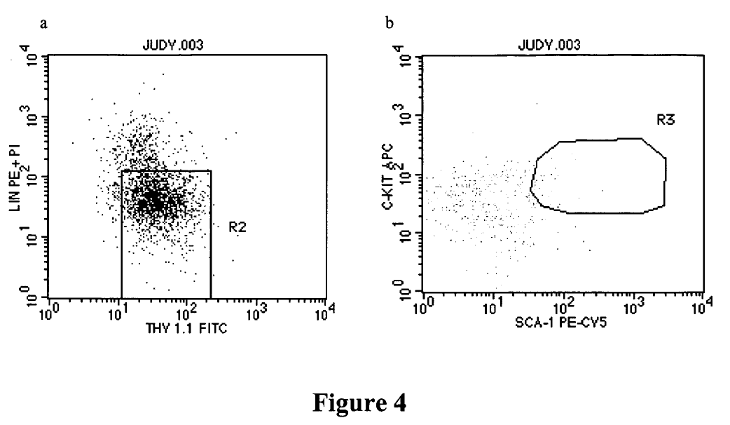

In order to investigate if the proliferating cells retain a stem cell phenotype, the cells were fixed and analyzed for HSC cell-surface markers. The result shows that most of the cells maintained the HSC characteristics at a phenotypic level (i.e. they are Lin negative, Sca-1 positive (low), C-kit postive, and Thy-1.1 positive (FIG. 4).

### EXAMPLE 2

### Expression of Adiponectin Receptors in Proliferating HSCs

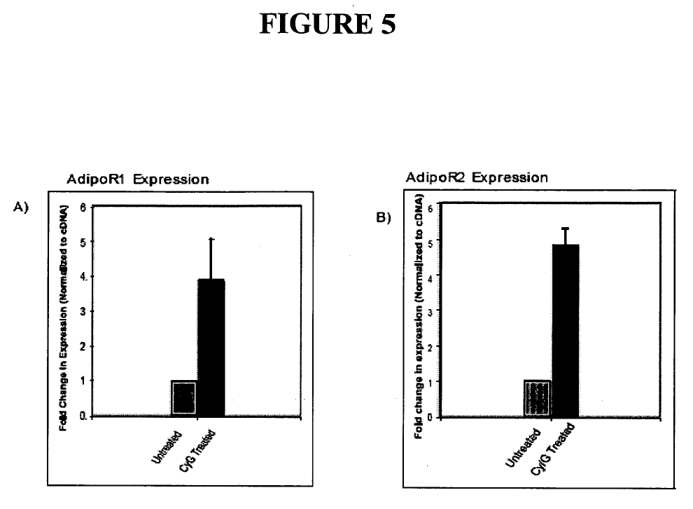

This Examples describes assays used to determine the levels of Adiponectin Receptor expression levels in proliferating HSCs. To determine whether an increase in AdipoR1 occurs when HSCs regenerate in vivo following Cy/G induced damage, the relative levels of this receptor were examined by qPCR in quiescent KTLS cells isolated from untreated mice and in proliferating KTLS cells isolated immediately following Cy/G treatment. It was determined that expression of AdipoR1 was upregulated approximately three fold in proliferating KTLS cells in vivo (FIG. 5a). Additionally, a similar pattern was determined for the AdipoR2 (FIG. 5b), a second receptor for Adipoenctin, and highly homologous to AdipoR1. Furthermore, Adiponectin, the ligand for both AdipoR1 and R2 was expressed in whole bone marrow stromal cell populations, as well as primary osteoblasts, bone forming cells which have recently shown to be part of the hematopoietic stem cell niche. These data indicate that upregulation of Adiponectin receptor and subsequent activation of Adiponectin signaling is involved in inducing or enhancing HSC growth.

### EXAMPLE 3

### Affect of Adiponectin on HSCs

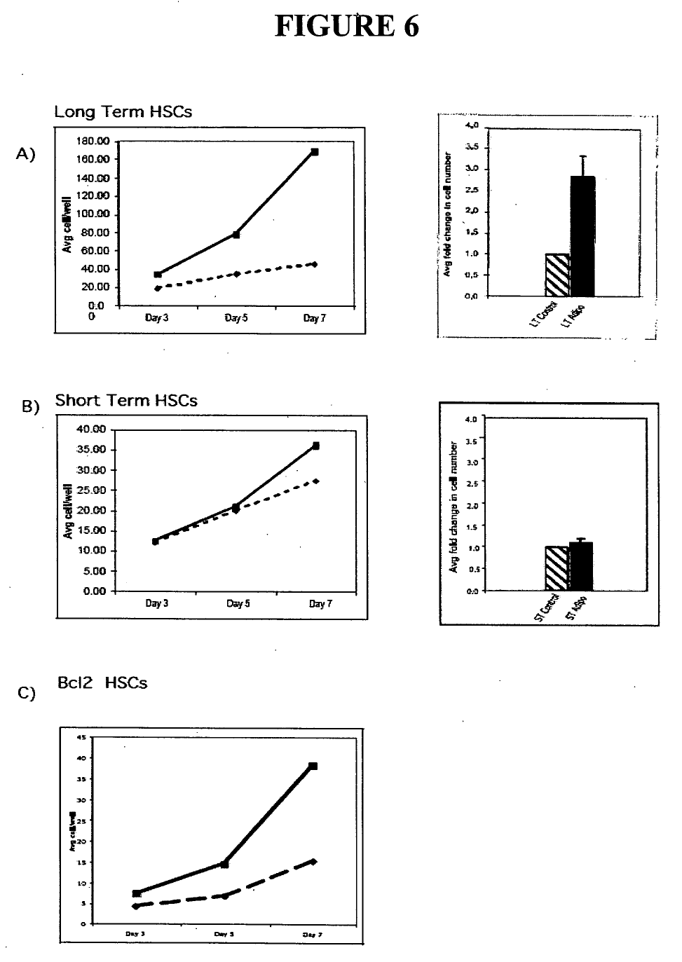

This Example describes the characterization of HSCs after being exposed to recombinant adiponectin. In particular, this Examples examined the effect of recombinant Adiponectin on both long term HSCs (KTSLin⁻) and short term HSCs (KTSLin^{lo}). Freshly isolated KTLS cells were cultured in serum free conditions with Adiponectin or vehicle control along with limiting doses of the cytokines SCF and Tpo. The presence of Adiponectin led to an increase in KTSLin⁻ cell number that ranged from two to four fold over control cell number (FIG. 6a). In contrast, no significant change was observed in the proliferation of KTSLin^{lo} (FIG. 6b) in response to Adiponectin. Furthermore the use of Bcl2 transgenic KTSLin⁻ cells allowed proliferation to occur in the presence of SCF and Adiponectin alone under serumfree conditions (FIG. 6c). These data show that adiponectin signaling not only induces proliferation of KTLS cells, but also that it preferentially activates the more quiescent long term subpopulation.

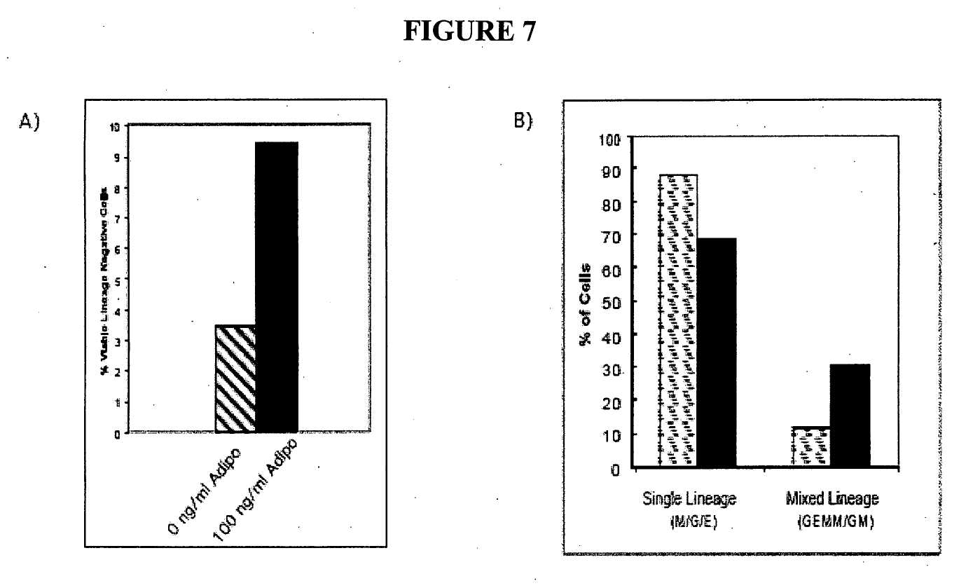

In order to further characterize the cells expanded in response to adiponectin, the cells were stained following seven to eight days of culture for surface expression of lineage markers. As shown in FIG. 7a, an average of three fold more adiponectin cultured cells maintained a lineage negative phenotype in comparison to control cultured cells. To test whether these cells retained progenitor cell function, the in vitro colony forming ability of cells cultured in the presence or absence of adiponectin were compared. In this culture system, the generation of single-lineage colonies is indicative of a committed cell type, whereas multilineage colonies are indicative of a more immature cell type having the ability to differentiate into various lineages. Cells cultured in the presence of adiponectin generated three fold more multilineage colonies when compared to cells cultured without Adiponectin, indicating enhanced maintenance of a functionally immature state in vitro.

### EXAMPLE 4

### Reduced Adiponectin Signaling Impairs HSC Proliferation

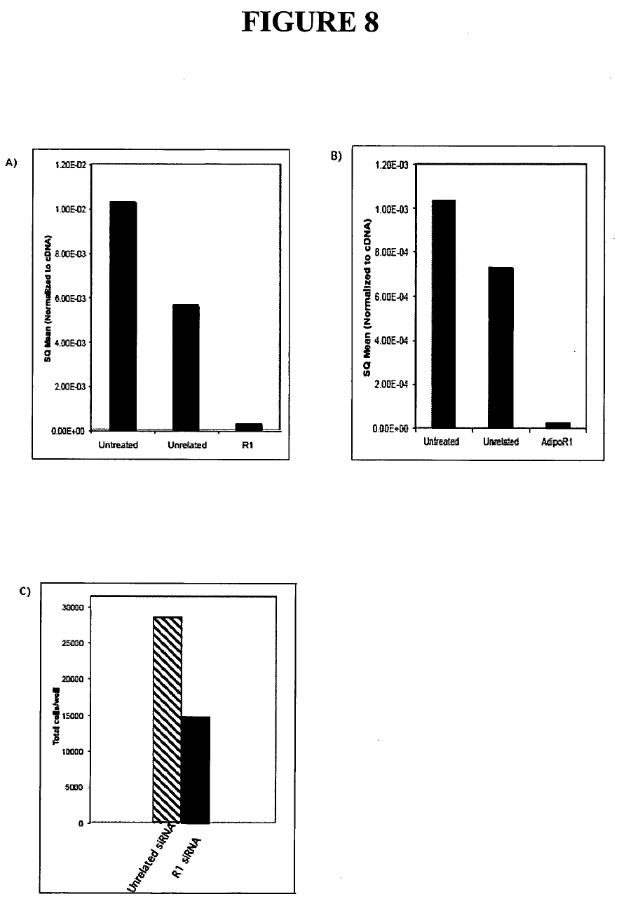

This Example demonstrates that reduced adiponectin signaling impairs HSC proliferation. siRNAs were generated against adiponectin's receptor, AdipoR1, which is more highly expressed in HSCs. A viral siRNA system was utilized in which expression of the siRNA target sequence is driven by a U6 promoter from a lentiviral vector containing an independently driven GFP sequence allowing infected cells to be visualized and analyzed. To test the efficiency of this siRNA, a 7F2 osteoblast cell line was infected with the lentiviral vector containing AdipoR1 siRNA or an unrelated (LacZ) control siRNA. GFP+ cells were sorted and AdipoR1 expression was analyzed by qPCR. Infection with the AdipoR1 siRNA resulted in a 90% knockdown in AdipoR1 expression as determined by real time PCR analysis (FIG. 8a). Similarly, infection of KTLS cells with AdipoR1 siRNA led to a 90% knockdown of AdipoR1 expression (FIG. 8b). To test the effect of Adiponectin receptor knockdown on HSC growth, HSCs were cultured in rich growth media (2% FBS, 30 ng/ml SCF, 30 ng/ml Flt3). Cells in which AdipoR1 expression was knocked down exhibited a two fold decrease in total cell number after 4 days in culture (FIG. 8c). In order to examine whether the decrease in cell number was due to a proliferation defect or increased death in the absence of AdipoR1, the rate of cell cycle entry of these cells was examined. It was determined that while cell death was unaffected by AdipoR1 knock down (FIG. 8d) cells with knocked down expression of AdipoR1, entered cycle at a lower rate than control cells (FIG. 8d). This loss of function data indicates that at least in vitro, activation of this pathway is important for maintaining normal growth responses of HSCs.

### EXAMPLE 5

### Adiponectin Leads to Activation of Pathways Involved in Metabolism in HSCs

This Example describes assay used to determine if adiponectin causes increase phosphorylation of AMP activated protein kinase. The primary pathways through which adiponectin functions is by phosphorylation of AMP activated protein kinase, which inhibits acetyl CoA carboxylase and increases fatty acid beta-oxidation. KTLS cells were treated with adiponectin or vehicle control, and phosphorylation of AMP kinase was monitored. While phosphorylation of AMP kinase was low in the presence of control vehicle stimulation AMP kinase was highly phosphorylated in nearly 100% of KTLS cells within a period of 30 minutes. As a positive control, HSCs were stimulated with AICAR a synthetic activator of AMP kinase.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

We claim:

1. A method for in vitro expansion of stem cells comprising:
a) providing:
i) stem cells; and
ii) a composition comprising adiponectin or an adiponectin variant; and
b) culturing said stem cells with said composition under conditions that promote expansion of said stem cells.

2. The method of claim 1, wherein said culturing generates a population of expanded stem cells.

3. The method of claim 1, further comprising providing a host and transplanting at least a portion of said population of expanded stem cells into said host.

4. The method of claim 3, wherein said stem cells are provided from said host.

5. The method of claim 1, wherein said composition comprises adiponectin.

6. The method of claim 1, wherein said culturing is under conditions whereby said cells retain their pluripotential phenotype after expansion.

7. The method of claim 1, wherein said stem cells are hematopoitic stem cells.

8. The method of claim 1, wherein said stem cells are selected from the group consisting of stromal stem cells, neuronal crest stem cells, mesenchymal stem cells, pulmonary stem cells and hepatic stem cells.

9. The method of claim 1, wherein said composition comprises at least one additional growth factor.

10. The method of claim 1, wherein said composition comprises a growth factor selected from the group consisting of SCF, FLT-3, and Tpo.

11. The method of claim 1, wherein said stem cells comprise human stem cells.

12. The method of claim 1, wherein said adiponectin or adiponectin variant is recombinant.

13. The method of claim 1, wherein said stem cells are ex vivo sorted stem cells.

14. The method of claim 1, wherein said adiponectin or adiponectin variant is present at a concentration of between 80 and 120 ng/ml.

15. The method of claim 1, wherein said adiponectin or adiponectin variant is present at a concentration of between 90 and 110 ng/ml.

16. A composition comprising;
a) ex vivo sorted stem cells; and
b) adiponectin or an adiponectin variant present in a concentration between 50 and 150 ng/ml.

17. The composition of claim 16, wherein said composition comprises at least one additional growth factor.

18. The composition of claim 16, wherein said at least one additional growth factor is present at a concentration between 5 ng/ml and 50 ng/ml.

19. The composition of claim 16, wherein said adiponectin or adiponectin variant is present at a concentration of between 80 and 120 ng/ml.

20. A kit comprising;
a) a composition comprising adiponectin or an adiponectin variant; and
b) written instructions for using said composition to expand stem cells.
